(19) United States
(12) Patent Application Publication
(10) Pub. No.: US 2014/0135752 A1
(43) Pub. Date: May 15, 2014

(54) System and Method for Configuring Fragmentation Segments of a Crystalline Lens for a Lensectomy

(71) Applicants: Vladislav Duma, Muenchen (DE); Toh Seng Goh, Wildwood, MO (US); Brian D. McCary, Clayton, MO (US); Gwillem Mosedale, Muenchen (DE)

(72) Inventors: Vladislav Duma, Muenchen (DE); Toh Seng Goh, Wildwood, MO (US); Brian D. McCary, Clayton, MO (US); Gwillem Mosedale, Muenchen (DE)

(21) Appl. No.: 13/837,112

(22) Filed: Mar. 15, 2013

Related U.S. Application Data
(63) Continuation-in-part of application No. 13/795,641, filed on Mar. 12, 2013.
(60) Provisional application No. 61/724,729, filed on Nov. 9, 2012.

(51) Int. Cl. A61F 9/008; A61F 9/007

(52) U.S. Cl. CPC A61F 9/00825; A61F 9/00736
USPC 606/6

(57) ABSTRACT

A system and method are provided for fragmenting a crystalline lens inside an operational volume in the lens. A reference axis that is based on the crystalline lens is established, and various pluralities of parallel planes are defined relative to the reference axis. A pulsed laser beam is then used to cut tissue in selected planes, to thereby fragment the lens by performing Laser Induced Optical Breakdown (LIOB) on the tissue. In accordance with the present invention each plurality of parallel planes is characterized by a particular inclination angle “φ” relative to the reference axis, a particular azimuthal angle “θ” relative to the reference axis, and an intersection angle “ψ” relative to each other.

This application is a continuation-in-part of application Ser. No. 13/795,641 filed Mar. 12, 2013, which is currently pending and which claims the benefit of U.S. Provisional Patent Application Ser. No. 61/724,729, filed Nov. 9, 2012. The entire contents of application Ser. Nos. 13/795,641 and 61/724,729 are hereby incorporated by reference herein.

### FIELD OF THE INVENTION

The present invention pertains generally to systems and methods for fragmenting a crystalline lens in preparation for a lens removal surgery. More particularly, the present invention pertains to systems and methods which use a pulsed laser beam to fragment a crystalline lens. The present invention is particularly, but not exclusively, useful as a system and method for creating lens fragments that are geometrically shaped (configured), and are appropriately dimensioned to facilitate their removal from the capsular bag of an eye during cataract surgery.

### BACKGROUND OF THE INVENTION

A removal of the crystalline lens from its capsular bag inside an eye (i.e. a lensectomy) must necessarily be accomplished with minimum trauma to the eye. An important consideration in this regard is the preparation of the lens for such a removal. Heretofore, a common method that has been used to prepare a lens for removal has been to first emulsify the lens using ultrasonic techniques. The emulsified lens is then aspirated from the capsular bag. Lens preparation by ultrasonic emulsification, however, can be relatively labor intensive, and can introduce excessive energy into the eye, which causes unnecessary trauma. With this in mind, the use of lasers for the purpose of preparing a crystalline lens for a lensectomy poses an effective alternative for such a procedure.

It is well known that laser systems can be effectively used for various ophthalmic procedures. Like all surgical procedures, however, each laser procedure has its own particular challenge. In the case of a lens removal, the challenge is essentially two-fold. For one, the lens fragments that are created during a laser procedure must be appropriately dimensioned for subsequent aspiration using conventional surgical implements (e.g. well known phaco-needles). For another, the lens fragments must be geometrically shaped or configured to facilitate their removal from the lens capsule. On this point, it is particularly desirable that the configuration or shape of lens fragments be such as to not block or impede the removal of other lens fragments. Stated differently, it is desirable if the lens fragments can be shaped to actually promote or induce the removal of other lens fragments during their aspiration.

In light of the above, it is an object of the present invention to provide a system and method for performing a fragmentation wherein lens fragments are created and configured by a laser unit to facilitate the removal of the lens fragments during a subsequent aspiration. Another object of the present invention is to provide a system and method for employing a laser unit to perform a fragmentation procedure which reduces labor-intensive requirements, and which minimizes trauma to the eye during the procedure. Yet another object of the present invention is to provide a system and method for employing a laser unit to perform a cataract surgery procedure which is easy to implement, is simple to use and is comparatively cost effective.

### SUMMARY OF THE INVENTION

In accordance with the present invention, a system and method are provided for fragmenting the crystalline lens of an eye in preparation for a lens removal. In particular, this fragmentation is performed within an operational volume that is defined inside the lens. More specifically, the operational volume is defined with data that is collected by an imaging unit, such as a well-known Optical Coherence Tomography (OCT) device.

In preparation for a procedure, a reference axis is established using data that is obtained by the imaging unit and is based on the crystalline lens. Next, various pluralities (i.e. sets) of parallel planes are defined relative to the reference axis. In accordance with the present invention each plurality of parallel planes is characterized by a particular inclination angle “φ” relative to the reference axis, and a particular azimuthal angle “θ” relative to the reference axis. Also, an intersection angle, “ψ”, can be established between different pluralities (sets) of planes.

Once the different pluralities of parallel planes have been defined and oriented, a pulsed laser beam is used to cut tissue in selected planes. Preferably, the laser beam is configured to fragment the lens by performing Laser Induced Optical Breakdown (LIOB) on tissue in the lens.

For a first embodiment of the present invention, three different pluralities (i.e. sets) of parallel planes are identified in the operational volume. In this case, a first plurality and a second plurality of the planes intersect each other at an angle “ψ”. The third plurality then intersects both the first and second pluralities. In their relationship with the reference axis, a normal to each plurality (i.e. plane set) is coplanar with the reference axis and it is inclined at an angle “φ” relative to the reference axis. Preferably, the respective inclination angles “φ₁”, “φ₂” and “φ₃” are each in a range between approximately 20° and 60°. Also, in addition to its unique inclination angle “φ”, the normal to each plurality of parallel planes is also oriented at a unique azimuthal angle “θ” relative to the reference axis.

As envisioned for the present invention, along with the basic three sets of parallel planes mentioned above, a fourth plurality of parallel planes can be defined to intersect at least one other plurality of parallel planes. Further, a fifth plurality of parallel planes can be defined to also intersect at least one other plurality of parallel planes. Thus, the inclusion of additional sets of parallel planes can be continued at selected locations in the operational volume, as desired.

A second embodiment of the present invention is actually a more specific configuration for the first embodiment. In this second embodiment, however, only three sets (pluralities) of parallel planes are specifically required. In detail, each plane in the first plurality is inclined at a tilt angle “φ₁” relative to the reference axis. Similarly, each plane in the second plurality is inclined at a tilt angle “φ₂” relative to the reference axis, and each plane in the third plurality is inclined at a tilt angle “φ₃” relative to the reference axis. More specifically, the three inclination angles (φ₁, φ₂ and φ₃), and their respective azimuthal angles (θ₁, θ₂ and θ₃) are selected to create symmetrical fragments with rectangular shaped sides.

A preferred result for the configuration of this second embodiment is a cube having square sides. To do this, φ₁=45° with θ₁=0°; φ₂=45° with θ₂=120°; and φ₃=45° with θ₃=240°. A beneficial consequence of this arrangement is that the fragment cubes are all aligned with a respective diagonal that is parallel to the reference axis. The diagonal in this case is a line that extends through the cube, between opposite corners of the cube.

For a third embodiment of the present invention, at least four sets (pluralities) of parallel planes are involved. In this case, rather than a cube, an octahedron is the result. For purposes of discussion, these four sets of planes are based on points in the operational volume that can be identified using Cartesian coordinates (n, m, p). Respectively, for each coordinate; n=0→n, m=0→m, and p=0→p.

In order to set up this third embodiment, five points are identified relative to the reference axis, beginning with a start point (i.e. a first point) at the coordinates (n, m, p). A second point at (n+1, m, p), a third point at (n, m+1, p), a fourth point at (n+1, m+1, p) and a fifth point at (n+0.5, m+0.5, and p±Δ) are then identified. Based on these points, a first plurality of parallel planes can be established from the first, second and fifth points. A second plurality of parallel planes can be defined from the second, third and fifth points. A third plurality of parallel planes can be defined from the third, fourth and fifth points. And, a fourth plurality of parallel planes can be defined from the fourth, first and fifth points. As will be appreciated, this results in an octahedron having an upper section (p=+Δ) and a lower section (p=−Δ). The resultant upper and lower sections are separated from each other by a base plane that is collectively defined by the first, second, third and fourth points. Within this structure, the upper section may be separated from the lower section, at the base plane, by a distance equal to a predetermined value of “p” to, in effect, establish an elongated, extended, or stretched, octahedron.

For all of the embodiments disclosed above, tissue cutting can be accomplished by disrupting tissue along lines that are established at the intersections of a cutting plane and a selected plurality of the parallel planes. Preferably, the cutting plane will be oriented perpendicular to the reference axis, and the disruption (cutting) of tissue is accomplished using a pulsed laser beam to disrupt the tissue by causing Laser Induced Optical Breakdown (LIOB) of the tissue. In more detail, LIOB is accomplished sequentially as successive cutting planes are advanced in an anterior direction.

From a system perspective, the present invention includes an imaging unit (e.g. OCT device) for collecting data that can be used to define the reference axis based on the lens. The imaging unit may also collect data that can be used to establish the operational volume in the crystalline lens. Also included in the system is a computer that is connected to receive input from the imaging unit. With this connection, in addition to the reference axis and the operational volume, the computer uses operator input to define the various pluralities of parallel planes, as well as their orientations in inclination angle “φ” and azimuth angle “θ” relative to the reference axis. Additionally, the system of the present invention will include a laser unit that is controlled by the computer to generate a pulsed laser beam for cutting tissue by Laser Induced Optical Breakdown (LIOB) of the tissue on selected planes in the first, second and third pluralities to create fragments of the crystalline lens.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic presentation of operative components of a system for the present invention;

FIG. 2 is a perspective view of a crystalline lens of an eye with a defined operational volume for the present invention, shown in phantom;

FIG. 3 is a top plan view of intersection parallel planes for use in defining lens fragments in accordance with the present invention;

FIG. 4 is a three-dimensional perspective view of a lens fragment generated with the intersection planes shown in FIG. 3;

FIG. 5 is a cube shaped lens fragment shown with reference indicators for orienting the lens fragment in a three-dimensional space within the operational volume shown in FIG. 2;

FIG. 6 is a presentation of the cube shaped lens fragment shown in FIG. 5 with cutting lines identified on the surfaces of the lens fragment;

FIG. 7 is a perspective view of a portion of an octahedral lens fragment shown with operational orientation points as envisioned for the present invention;

FIG. 8 is a perspective view of octahedral shaped lens fragments as generated for the present invention; and

FIG. 9 is an elongated (stretched) version of the octahedral shaped lens fragments presented in FIG. 8.

### DESCRIPTION OF THE PREFERRED EMBODIMENTS

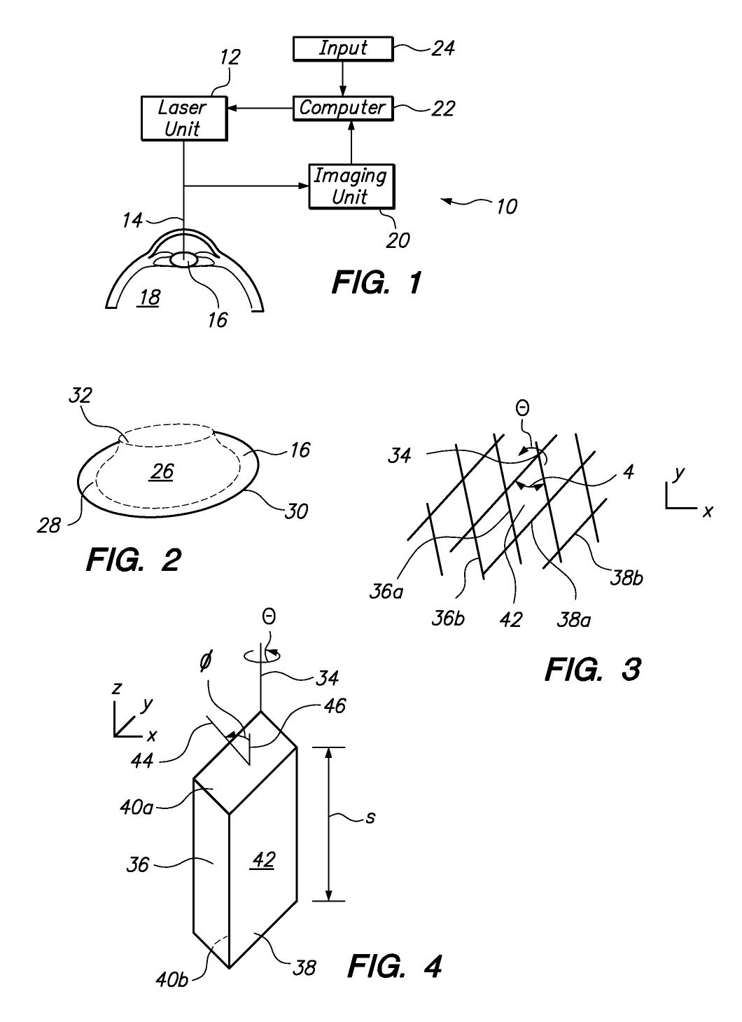

Referring initially to FIG. 1, a system for fragmenting tissue in accordance with the present invention is shown and is generally designated 10. As shown, the system 10 includes a laser unit 12 for generating a pulsed laser beam (e.g. a so-called femtosecond laser), and for directing the laser beam along a beam path 14 toward the crystalline lens 16 of an eye 18. Also included in the system 10 are an imaging unit 20 (e.g. an OCT imaging device) and a computer 22. For the present invention, the imaging unit 20 receives imaged data from the crystalline lens 16, and then passes this data as input to the computer 22. The computer 22 also receives operator-generated input 24 that includes operational parameters, and uses this input 24, together with the data from the imaging unit 20, to control the laser unit 12.

As best appreciated with reference to FIG. 2, the tissue of interest for the present invention is located inside the crystalline lens 16. More specifically, the target tissue is within an operational volume 26 (shown in phantom), and this operational volume 26 is, at least for the most part, separated by a safety margin 28 from the capsular bag 30 of the crystalline lens 16. The exception here is the anterior portion 32 of the capsular bag 30 where the opening is established for eventually removing the crystalline lens 16 from the capsular bag 30. In the event, the safety margin 28 will typically extend approximately fifty microns, or more, from the capsular bag 30 into the crystalline lens 16.

FIG. 3 shows a generalized layout plan for operating the system 10 of the present invention. In detail, FIG. 3 shows an end-on view of a reference axis 34 as established for the present invention, along with a plurality of parallel planes, i.e. a set 36, of which the planes 36a and 36b are exemplary. Another plurality of parallel planes, i.e. a set 38, of which the planes 38a and 38b are exemplary, is also shown in FIG. 3. In their relationship with each other, the plane sets 36 and 38 will intersect each other at an angle “ψ”, which may be either a right angle or an acute angle.

In FIG. 4 it will be seen that a third plurality of parallel planes are also defined for the present invention, i.e. a set 40, of which the planes 40a and 40b are exemplary. Together, these three plane sets 36, 38 and 40 establish a three dimensional lens fragment 42. As will be appreciated by the skilled artisan, the present invention envisions that a plurality of contiguous lens fragments 42 can be defined that will extend throughout the operational volume 26, or selected portions thereof.

Still referring to FIGS. 3 and 4, it will be noted that, although the parallel plane sets 36 and 38 are each respectively parallel to the reference axis 34, the parallel plane set 40 is not. Instead, set 40 intersects the reference axis 34. Further, FIG. 4 shows that the parallel plane set 40 is inclined to the reference axis 34 by a tilt angle “φ”. Specifically, the tilt angle “φ” is identified as the angle between a normal 44 to the surface of a plane in the plane set 40, and a parallel reference axis 46, i.e. an axis that intersects the normal 44 and is parallel to the reference axis 34. FIG. 4 also indicates that the lens fragment 42 can be selectively positioned at a predetermined azimuthal angle “θ” relative to the reference axis 34, and that the distance “s” between adjacent planes in a plane set 36, 38 or 40 may be different and can be established as is necessary or desirable.

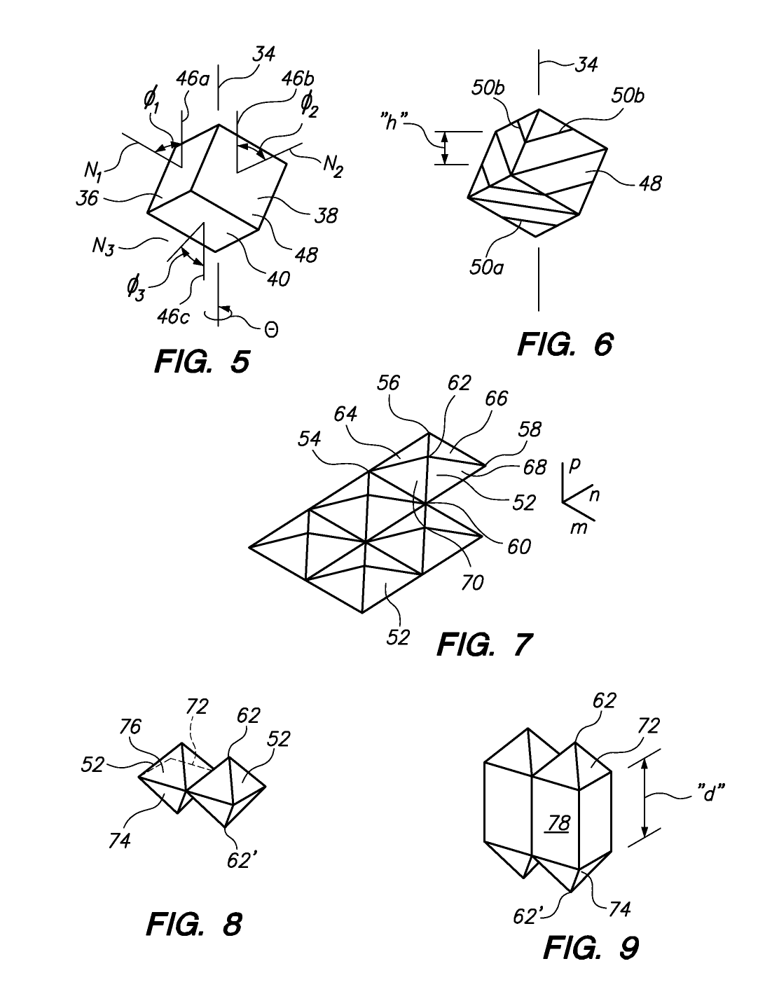

A preferred embodiment of the present invention is shown in FIG. 5 as a variant of the lens fragment 42 (see FIG. 4). In this preferred embodiment, the lens fragment 42 will have the specific configuration of a cube 48. In this embodiment, all of the plane sets 36, 38 and 40 intersect the reference axis 34. Accordingly, each plane in the set 36 is inclined at a same tilt angle “φ₁” relative to the reference axis 34 (i.e. the angle between a normal “N₁” and the parallel reference axis 46a). Similarly, each plane in the set 38 is inclined at a same tilt angle “φ₂” relative to the reference axis 34 (i.e. the angle between a normal “N₂” and the parallel reference axis 46b). And, likewise, each plane in the set 40 is inclined at a same tilt angle “φ₃” relative to the reference axis 34 (i.e. the angle between a normal “N₃” and the parallel reference axis 46c). Moreover, each side of the cube 48 is square, all of the tilt angles “φ” are equal to each other (φ₁=φ₂=φ₃), and each of the sets 36, 38 and 40 will have a respectively prescribed azimuthal angle “θ”. Specifically, the orientation of each contiguous cube 48 in the operational volume 26 will be such that φ₁=45° and θ₁=0°; φ₂=45° and θ₂=120°; and φ₃=45° and θ₃=240°.

FIG. 6 shows the cube 48, as presented in FIG. 5, with a plurality of intersection lines 50 indicated on the surface of the cube 48; of these, the intersection lines 50a and 50b are exemplary. In each instance, an intersection line 50 is established wherever a cutting plane intersects with the surface of the cube 48, or with any other defined lens fragment (e.g. lens fragment 42). Stated differently, a cutting plane is also indirectly designated by respective intersection lines 50. With this in mind, FIG. 6 also indicates there will be a vertical distance “h” between adjacent intersection lines 50 (i.e. adjacent cutting planes). Specifically, “h” is measured parallel to the reference axis 34 and, for purposes of the present invention, the distance “h” will preferably be in a range of around 5-20 microns.

Another embodiment for generating lens fragments in accordance with the system 10 of the present invention is presented in FIGS. 7, 8 and 9. The result in this case is a plurality of octahedral shaped lens fragments 52. To geometrically define an octahedral shaped lens fragment 52, it is necessary to identify at least five points in the operational volume 26. In particular, each point will have respective Cartesian coordinates (n, m, p); where n=0→n, m=0→m, and p=0→p. In FIG. 7, beginning with a first point 54 at (n, m, p), a second point 56 at (n+1, m, p), a third point 58 at (n+1, m+1, p), a fourth point 60 at (n, m+1, p) and a fifth point 62 at (n+0.5, m+0.5, and p±4) are identified. Different sets of parallel planes can then be defined using these points. Specifically, a plurality of parallel planes (i.e. plane set 64) is defined by the points 54, 56 and 62; a different plurality of parallel planes (i.e. plane set 66) is defined by the points 56, 58 and 62; another plurality of parallel planes (i.e. plane set 68) is defined by the points 58, 60 and 62; and a plurality of parallel planes (i.e. plane set 70) is defined by points 60, 54 and 62.

FIG. 8 shows that the above defined plane sets 64, 66, 68 and 70 will collectively result in an octahedral shaped lens fragment 52 having an upper section 72 (p=+Δ) and a lower section 74 (p=−Δ). In this case, the upper section 72 (using point 62) and the lower section 74 (using point 62′) are separated by a base plane 76. At this point it is noted that the base plane 76 may be incised, creating inverted pyramids. Using this same general construction, an alternate embodiment of this configuration is shown in FIG. 9. For this alternate embodiment it is shown that the upper section 72 is separated from the lower section 74, at the base plane 76, by a distance equal to a predetermined value of “d”, to create an elongated octahedron 78.

In an operation of the system 10, the imaging unit 20 provides input to the computer 22 for defining the reference axis 34 relative to the eye 18, and for establishing the operational volume 26 in the crystalline lens 16. Also, the input 24 that is provided to the computer 22 by the system operator (not shown) will include operational parameters, such as φ, θ, ψ, s, h and d, for defining various sets of parallel planes (e.g. set 36). Using this information, the laser unit 12 is controlled by the computer 22 to generate a pulsed femtosecond laser beam for use in cutting tissue in the operational volume 26. Specifically, this cutting is caused by a Laser Induced Optical Breakdown (LIOB) of the tissue along selected intersection lines 50.

In detail, the LIOB of tissue is accomplished in a cutting plane along intersection lines 50. Preferably, the cutting plane is perpendicular to the reference axis 34. Further, as envisioned for the present invention, the LIOB of tissue will be accomplished sequentially as successive cutting planes are advanced through the operational volume 26 in an anterior direction.

Referring back to FIG. 1, and considering the present invention from a system perspective, it will be appreciated that the computer 22 is a centrally essential component of the system. Most importantly, the computer 22 effectively controls the laser unit 12 in the conduct of the system's methodologies, and it does so using both system-internal inputs from the imaging unit 20 and external inputs 24 from a user (not shown). A computer program product is therefore required to effectively coordinate the use of these inputs 24 for an operation of the laser unit 12.

Structurally, as shown in FIG. 1, the system 10 requires electronic interconnections between the laser unit 12, the imaging unit 20 and the computer 22. In detail, the system 10 uses the imaging unit 20 to define a reference axis 34 that is based on the eye 18 (e.g. the lens 16). The system 10 also uses the imaging unit 20 for identifying the operational volume 26 in the crystalline lens 16. All of this is accomplished using the so-called system-internal inputs mentioned above.

Insofar as the external inputs 24 are concerned, they are primarily geometric and operational parameters provided by the user to operate the laser unit 12 for fragmentation of the crystalline lens 16. Specifically, the inputs 24 will include details for configuring the laser beam to perform LIOB, and it will include the geometric parameters (e.g. φ, θ, ψ, h and d) that are necessary for defining the fragments 42 (e.g. cube and octahedral) that are to be removed from the eye 18.

In overview, the computer 22 is operationally pre-programmed with a computer program product having program sections that will result in defining fragments 42 that are created between different pluralities of parallel planes. For example, the input 24 can provide parameters for defining a first plurality of parallel planes; a second plurality of parallel planes, wherein the first and second pluralities intersect each other at an angle “ψ”; and for defining a third plurality of parallel planes, wherein the third plurality intersects both the first and second pluralities to create fragments 42. The alignment of fragments 42 in the operational volume 26 can be further refined with greater specificity by defining their orientation in the operational volume 26. This is done by selecting a normal to one of the sets of planes which is coplanar with the reference axis 34, and is inclined at an angle “φ” relative to the reference axis 34. A computer program section can then manipulate the tilt angle “φ”, and the azimuth angle “θ”, to simultaneously orient all of the fragments 42 as specifically desired by the user.

For a preferred embodiment of the present invention, the fragments 42 are created as “cubes”. In this case, the computer 22 includes program sections which will establish that each first, second and third pluralities will be respectively inclined at a tilt angle “φ₁”, “φ₂”, and “φ₃” relative to the reference axis 34. Further, for this embodiment, “φ₁”, “φ₂” and “φ₃” will all be 45°, and each side of the fragment 42 will be square. As envisioned for the present invention, this arrangement results in each fragment 42 (i.e. “cube”) being oriented with an internal diagonal aligned parallel to the reference axis 34.

For specific instances wherein it is desired to have fragments 42 formed as octahedrons, a program section for the computer 22 can be included that will first locate a start point on the reference axis 34. The program section will then be used to identify five points in the operational volume 26 having respective Cartesian coordinates (n, m, p) relative to a start point. Specifically, computer 22 can be programmed to have n=0→n, m=0→m, and p=0→p.

Using Cartesian coordinates, a program section for the computer 22 can be programmed to define octahedral shaped fragments 42. In detail, an upper section 72 and a lower section 74 for the octahedron are defined with a first point at (n, m, p), a second point at (n+1, m, p), a third point at (n, m+1, p), a fourth point at (n+1, m+1, p) and a fifth point at (n+0.5, m+0.5, and p±Δ). Several pluralities of parallel planes can then be defined using these points. Specifically, a first plurality of parallel planes is defined using the first, second and fifth points; a second plurality of parallel planes is defined by the second, third and fifth points; a third plurality of parallel planes is defined by the third, fourth and fifth points; and a fourth plurality of parallel planes is defined by the fourth, first and fifth points. The result here is an octahedron having an upper section 72 (p=+Δ) and a lower section (p=−Δ).

For an octahedral shaped fragment 42, the upper section 72 and the lower section 74 are separated by a base plane 76 that is defined by the first, second, third and fourth points. As a variation of this embodiment, the upper section 72 and the lower section 74 can be separated from each other at the base plane 76 by a distance “d”. With this in mind, two configurations are possible. For one, the separation distance “d” may be zero. If so, the incision of tissue can be made directly on the base plane 76. In another instance, the separation distance “d” may be greater than zero. In this case, the incision of tissue can occur on a cutting plane that is parallel to the base plane 76, wherein the cutting plane is intermediate the upper section 72 and the lower section 74.

It will be further appreciated by the skilled artisan that for any of the above embodiments of the present invention, there can be any number of different sets of parallel planes. In each case, a program section of the computer 22 can be employed to establish the particular set of planes as a part of the methodology for the present invention. In the event interstitials are formed during an operation of the present invention, it is envisioned that they will be dimensioned during fragmentation of the crystalline lens 16 to be sufficiently diminutive for appropriate removal with the fragments 42. For an operation of the present invention, the laser unit 12 will be controlled by the computer 22 to disrupt tissue along lines established by the intersections of a cutting plane and a selected plurality of the parallel planes. As specifically intended for the present invention, the disrupting of tissue in the operational volume 26 will be accomplished using a pulsed laser beam to cause Laser Induced Optical Breakdown (LIOB) of the tissue.

While the particular System and Method for Configuring Fragmentation Segments of a Crystalline Lens for a Lensectomy as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A method for fragmenting a crystalline lens wherein the lens defines a reference axis, the method comprising the steps of:
establishing an operational volume in the crystalline lens;
defining a first plurality of first parallel planes;
defining a second plurality of second parallel planes, wherein the first and second pluralities intersect each other at an angle “ψ”;
defining a third plurality of third parallel planes, wherein the third plurality intersects both the first and second pluralities, and wherein a normal to a third plane is coplanar with the reference axis and is inclined at an angle “φ” relative thereto; and
cutting tissue on selected planes in the first, second and third pluralities to create fragments of the crystalline lens.

2. A method as recited in claim 1 further comprising the step of defining a fourth plurality of fourth parallel planes, wherein the fourth plurality intersects at least one other plurality of parallel planes and the cutting step is accomplished on a selected plane in the fourth plurality.

3. A method as recited in claim 2 further comprising the steps of:
defining “n” different pluralities of parallel planes, wherein n>4, and each plurality intersects at least one other plurality of parallel planes, and the cutting step is accomplished on a selected plane in each plurality.

4. A method as recited in claim 1 wherein each first plane is inclined at a tilt angle “φ₁” relative to the reference axis, wherein each second plane is inclined at a tilt angle “φ₂” relative to the reference axis, and wherein each third plane is inclined at a tilt angle “φ₃” relative to the reference axis.

5. A method as recited in claim 4 wherein φ₁=φ₂=φ₃.

6. A method as recited in claim 4 wherein “φ₁”, “φ₂” and “φ₃” are each in a range between approximately 20° and 60°.

7. A method as recited in claim 1 wherein each first plane is inclined at a tilt angle “φ₁” relative to the reference axis, and a normal to each first plane lies in a same plane with the reference axis at an azimuthal angle “θ₁”, and wherein φ₁=45° and θ₁=0°; wherein each second plane is inclined at a tilt angle “φ₂” relative to the reference axis, and a normal to each second plane lies in a same plane with the reference axis at an azimuthal angle “θ₂”, and wherein φ₂=45° and θ₂=120°; and wherein each third plane is inclined at a tilt angle “φ₃” relative to the reference axis, and a normal to each third plane lies in a same plane with the reference axis at an azimuthal angle “θ₃”, and wherein φ₃=45° and θ₃=240°.

8. A method as recited in claim 1 wherein the cutting step further comprises the steps of:
disrupting tissue along lines established by the intersection of a cutting plane and a selected plurality of the parallel planes, wherein the cutting plane is perpendicular to the reference axis; and
sequentially performing the cutting step for lines in the cutting plane on successive first, second and third pluralities of parallel planes.

9. A method as recited in claim 8 wherein the disrupting step is accomplished using a pulsed laser beam to disrupt tissue by causing Laser Induced Optical Breakdown (LIOB) of the tissue.

10. A method as recited in claim 8 further comprising a plurality of cutting planes, wherein the cutting and performing steps are performed in cutting planes as successive cutting planes are advanced in an anterior direction.

11. A method as recited in claim 10 wherein the distance between adjacent cutting planes is “s”.

12. A method as recited in claim 1 wherein “ψ” is an acute angle.

13. A method for fragmenting a crystalline lens, wherein the lens defines a reference axis, the method comprising the steps of:
establishing an operational volume in the crystalline lens;
defining a first plurality of first parallel planes in the operational volume, wherein each first plane is inclined at a tilt angle “φ₁” relative to the reference axis, and a normal to each first plane lies in a same plane with the reference axis at an azimuthal angle “θₗ”, and wherein φ₁=45° and θ₁=0°;
defining a second plurality of second parallel planes in the operational volume, wherein each second plane is inclined at a tilt angle “φ₂” relative to the reference axis, and a normal to each second plane lies in a same plane with the reference axis at an azimuthal angle “θ₂”, and wherein φ₂=45° and θ₂=120°;
defining a third plurality of third planes in the operational volume, wherein each third plane is inclined at a tilt angle “φ₃” relative to the reference axis, and a normal to each third plane lies in a same plane with the reference axis at an azimuthal angle “θ₃”, and wherein φ₃=45° and θ₃=240°; and
cutting tissue on selected planes in the first, second and third pluralities to create fragments of the crystalline lens.

14. A method as recited in claim 13 wherein the cutting step further comprises the steps of:
disrupting tissue along lines established by the intersection of a cutting plane and a selected plurality of the parallel planes, wherein the cutting plane is perpendicular to the reference axis, and wherein the disrupting step is accomplished using a pulsed laser beam to disrupt tissue by causing Laser Induced Optical Breakdown (LIOB) of the tissue; and
sequentially performing the cutting step for lines in the cutting plane on successive first, second and third pluralities of parallel planes.

15. A method as recited in claim 14 further comprising a plurality of cutting planes, wherein the disrupting and performing steps are performed in cutting planes as successive cutting planes are advanced in an anterior direction.

16. A method for fragmenting a crystalline lens, wherein the lens defines a reference axis, the method comprising the steps of:
establishing an operational volume in the crystalline lens;
locating a start point on the reference axis;
identifying five points in the operational volume having respective Cartesian coordinates (n, m, p) relative to the start point, wherein n=0→n, m=0→m, and p=0→p, with a first point at (n, m, p), a second point at (n+1, m, p), a third point at (n, m+1, p), a fourth point at (n+1, m+1, p) and a fifth point at (n+0.5, m+0.5, and p±Δ);
defining a first plurality of first parallel planes in the operational volume, wherein one of the first parallel planes is defined by the first, second and fifth points;
defining a second plurality of second parallel planes in the operational volume, wherein one of the second parallel planes is defined by the second, third and fifth points;
defining a third plurality of third parallel planes in the operational volume, wherein one of the third parallel planes is defined by the third, fourth and fifth points;
defining a fourth plurality of fourth parallel planes in the operational volume, wherein one of the fourth parallel planes is defined by the fourth, first and fifth points; and
cutting tissue on selected planes in the first, second and third pluralities to create fragments of the crystalline lens.

17. A method as recited in claim 16 wherein the method results in an octahedron having an upper section (p=+Δ) and a lower section (p=−Δ), wherein the upper section and the lower section are separated by a base plane defined by the first, second, third and fourth points, and further wherein the upper section is separated from the lower section, at the base plane, by a distance “d” equal to a predetermined value.

18. A method as recited in claim 17 wherein “d” is zero and the cutting step is accomplished to include incising tissue on the base plane.

19. A method as recited in claim 17 wherein “d” is greater than zero and the cutting step is accomplished to include incising tissue on a cutting plane, wherein the cutting plane is parallel to the base plane and is intermediate the upper section and the lower section.

20. A method as recited in claim 16 wherein the cutting step further comprises the steps of:
disrupting tissue along lines established by the intersections of a cutting plane and a selected plurality of the parallel planes, wherein the cutting plane is perpendicular to the reference axis, and wherein the disrupting step is accomplished using a pulsed laser beam to disrupt tissue by causing Laser Induced Optical Breakdown (LIOB) of the tissue; and
sequentially performing the disrupting step for lines in the cutting plane on successive pluralities of parallel planes, and wherein there are a plurality of cutting planes, and the disrupting and performing steps are performed in cutting planes as successive cutting planes are advanced in an anterior direction.

21. A system for fragmenting a crystalline lens which comprises:
an imaging unit for locating a reference axis based on the lens, and for establishing an operational volume in the crystalline lens;
a computer connected to the imaging unit to receive input from the imaging unit for defining a first plurality of first parallel planes, for defining a second plurality of second parallel planes, wherein the first and second pluralities intersect each other at an angle “ψ”, and for defining a third plurality of third parallel planes, wherein the third plurality intersects both the first and second pluralities, and wherein a normal to a third plane is coplanar with the reference axis and is inclined at an angle “φ” relative thereto; and
a laser unit controlled by the computer to generate a pulsed laser beam for cutting tissue by causing a Laser Induced Optical Breakdown (LIOB) of the tissue on selected planes in the first, second and third pluralities to create fragments of the crystalline lens.

22. A system as recited in claim 21 wherein each first plane is inclined at a tilt angle “φ₁” relative to the reference axis, wherein each second plane is inclined at a tilt angle “φ₂” relative to the reference axis, and wherein each third plane is inclined at a tilt angle “φ₃” relative to the reference axis, and further wherein φ₁, φ₂ and φ₃ are each in a range between approximately 20° and 60°.
